(19) United States
(12) Patent Application Publication
(10) Pub. No.: US 2005/0143362 A1
(43) Pub. Date: Jun. 30, 2005

(54) Danazol for treatment of hypogonadism in the adult male

(76) Inventor: Michael W. McLane, Baltimore, MD (US)

Correspondence Address: Michael W. McLane, P.O. Box 39542, Baltimore, MD, 21212, US

(21) Appl. No.: 10/749,314

(22) Filed: Dec. 31, 2003

(51) Int. Cl. A61K 31/58; A61K 9/48

(52) U.S. Cl. 514/176; 424/451

(57) ABSTRACT

The method of use of danazol for enhancing the testosterone activity in the adult male. In particular, androgen activity levels are increased in the adult male suffering from hypogonadism by pharmaceutical compositions of danazol that are disclosed.

### FIELD OF THE INVENTION

The invention relates to danazol or a pharmaceutical composition thereof for treatment of hypogonadism in the adult male.

### INFORMATION DISCLOSURE STATEMENT

The Merk Index (Eleventh Edition, Merck & Co., Rahway, N.J., monograph 2811, 1989) describes danazol under that name, which is the generic name and by the chemical name 17.alpha.-pregn-4-en-20-yno[2,3-d]isoxazol-17-ol and sets forth the following information concerning biological properties and clinical utility thereof:

Anterior pituitary suppressant [sic]. Anabolic steroid derivative of ethisterone, q . . . nu . . . , With mild androgenic side effects (an impeded androgen) . . . . Clinical studies in women with endometriosis . . . Use in idiopathic thrombocytopenic purpura . . . in hemophilia . . .

Therap Cat: Antigonadotropin.

Physician's Desk Reference.RTM. (46th Edition, pp.2046-2047, 1992) describes danazol under the brand name DANOCRINE.RTM, which is a pharmaceutical composition of danazol in capsule form containing as active ingredient 50 milligrams, 100 milligrams, or 200 milligrams of danazol per capsule and as inactive ingredients benzyl alcohol, gelatin, lactose, magnesium stearate, parabens, sodium propionate, starch and talc, with indication for use in endometriosis, fibrocystic breast disease, and hereditary angioedema.

Gelfand, Jeffery A. et al., New England Journal of Medicine, V. 295, p.1444, 1 976.

“ . . . Danazol, a derivative of ethinyltestosterone, is mildly myogenic (anabolic), but has markedly attenuated androgenic potential (an “impeded” androgen). It produces dose-dependent reduction of serum gonadotropins, resulting in a concomitant decrease of the primary sex hormone.”

Potts et al., Drugs, V. 19, p. 321, 1980

“ . . . Danazol has weak, “impeded”, androgen-like activity as well as anabolic activity. The existing in vivo data are consistent with the inhibition of hypothalamus or pituitary function as the primary activity of danazol.”

Zurlo et al., Fertility & Sterility, V. 54, p. 64, 1990.

“ . . . Its primary pharmacological actions are gonadotropin inhibition, suppression of sex steroid synthesis by inhibiting gonadal steroidogenesis enzyme systems, and competitive binding to progesterone and androgen receptors. Based on reports of its first clinical trial in 1971, danazol was found to be effective in treating endometriosis . . . . Danazol has also been approved in the treatment of fibrocystic breast disease and hereditary angioedema. Other conditions for which the drug has been reported efficacious are gynecomastia, excessive menstrual blood loss, and alpha-1-antitrypsin deficiency. Danazol has also been shown to reduce secondary sex characteristics that accompany precocious puberty in females and has some activity as both male and female contraceptives. Finally, the drug has been shown to reduce the bleeding diathesis in hemophilia A and B and to increase the platelet count in idiopathic thrombocytopenia purpura . . . ”

### BACKGROUND OF INVENTION

Prior art concerning the pharmaceutical utility of danazol as a therapeutic for male hypogonadism has been in evidence since the 1980's but the expertise in the field consistently misinterpreted the accumulated published data. The universally accepted expert interpretations of danazol pharmacology up to 1993 still teaches away from the invention of danazol as a treatment for hypogonadism. As seen above, danazol has always been labelled an “impeded androgen” in the literature due to an early and long-lasting misinterpretation of the pharmacological and physiological studies. On Jun. 14th, 1993, I invented and wrote down the new use of danazol as a treatment for hypogonadism in the adult male by reinterpreting several different pharmacological studies from the 1970's and 1980's. Specifically, the new use invention for danazol stems from an insightful reinterpretation of published data that had been 5-20 years old. For instance:
1. Danazol's “impedent” androgenicity in the male was accepted early on and had been unquestioned by experts in the field for at least 25 years. An early misinterpretation of danazol's mechanism of action led pharmacologists in the field to discount danazol's androgenic and therapeutic potential in hypogonadism. Although misintepreted for the last twenty years, Dmowski et al. in 1971 (Fertility & Sterility, 22,pp 9-18) presented experimental evidence that at new low dosages danazol affects an increased and stable, long term anabolic response in whole males.
2. Danazol's true mechanism of action as a competitive displacer of testosterone from sex hormone binding globulin (SHBG) was published in 1981 in the study by Pugeat et al.
3. The primary importance of SHBG in determining active testosterone levels in the hypogonadal male body was published in the 1980's in studies by Macphee et al. and by Korenman et al.

The “non-obvious” new integration of these long published observations with more recent findings previous to 1993 forms the basis for my new use invention for danazol. Clearly, the “secondary rule of evidence for non-obviousness” is valid in that no other expert in the field had yet proposed said invention, having had similar access to the published data for a reasonable period of time.

Enclosed with this application is a packet of dated documentation, validating my date of invention and my invention process. Jun. 14, 1993 is the priority date of my new utility invention for using danazol as a new therapeutic for treating hypogonadism in the adult male.

The invention is a medical advance for the oral treatment of hypogonadism in the adult male.

### SUMMARY OF THE INVENTION

In a process aspect the invention is the method of enhancing the testosterone activity in the adult male which comprises administering to the male person an amount of 17.alpha.-pregn-4-en-20-yno[2,3-d]isoxazol-17-ol or a pharmaceutical composition thereof effective in treating hypogonadism in the adult male.

In a composition aspect the invention is a pharmaceutical composition of 17.alpha.-pregn-4-en-20-yno[2,3-d]isoxazol-17-ol for reducing or treating hypogonadism in an adult male suffering therefrom.

### DETAILED DESCRIPTION OF THE INVENTION

As shown by the above-cited Merck Index and Physician's Desk Reference.RTM. citations, danazol is a known chemical substance and a drug of commerce and is available by prescription. The pharmaceutical composition thereof of the invention is any composition wherein the active ingredient is danazol and the inactive ingredients are pharmaceutically acceptable and do not interfere with the purpose of treating hypogonadism in the adult male. The composition can be prepared for oral, topical, parenteral, or rectal administration and can be in solid or liquid dosage form including capsules, tablets, suppositories, solutions, suspensions, or emulsions. Conventional pharmaceutically acceptable vehicles and techniques are used in preparing these dosage forms. A composition for oral administration is preferred. The amount of danazol in each unit dosage is such that a reasonable number of unit dosages per day, preferably one to four, produce the effect of reducing and treating hypogonadism in an adult male suffering therefrom.

A pharmaceutical composition of the invention is packaged for sale or distribution in packages with instruction for use thereof for reducing or treating hypogonadism in an adult male suffering therefrom.

The capsules described in claim 5 and containing as active ingredient 10 milligrams, 25 milligrams, and 50 milligrams of danazol per capsule and as inactive ingredients benzyl alcohol, gelatin, lactose, magnesium stearate, parabens, sodium propionate, starch, and talc are suitable for carrying out the invention and would each be effective in the following examples wherein administration of one danazol capsule from one to four times per day to an adult male suffering from hypogonadism would be found effective in reducing or treating the hypogonadism.

Illustrative examples are below:

### EXAMPLE 1

A man of 50 years reports a lethargic and depressed mood. Blood tests show a hypogonadism in which total testosterone is borderline normal range but SHBG levels are higher than normal. Free testosterone levels are abnormally low and the patient is diagnosed as hypogonadal. Treatment with one 25 milligram danazol capsule three times daily would begin. After one month, he reports greater vigor and an improved mood. Also, blood tests would show that free testosterone has increased and total testosterone and SHBG levels have remained stable.

### EXAMPLE 2

A man of 61 years reports a general physical weakening and decreased libido. Blood tests show a high total testosterone level but SHBG levels are also in the very high range. Free testosterone levels are abnormally low and the patient is diagnosed as hypogonadal. Treatment with one 25 milligram danazol capsule twice a day would begin. After one month, patient reports an increased libido. Also, blood tests would show a new higher level in free testosterone and lower levels of SHBG.

### References Cited

### U.S. Patent Documents

| | | | |
|---|---|---|---|
| 4835146 | May, 1989 | Harrington | 514/176 |
| 4837212 | June, 1989 | Harrington | 514/176 |
| 4997653 | March, 1991 | Igarashi | 514/176 |
| 5340805 | August, 1994 | Harrington, Jr. | 514/178 |

### Other References

The Merck Index, Eleventh Edition, Merck & Co., Rahway, N.J., Monograph 2811, 1989.

Physicians Desk Reference.RTM., 46th edition, pp. 2046-2047, 1992.

Burke and Anderson, Nature, V. 240, pp. 38-40, 1972.

Dmowski et al., Fertility & Sterility, V. 22, pp. 9-18, 1971.

Gelfand et al., New England Journal of Medicine, V. 295, p. 1444, 1976.

Gershagen et al., Acta Obstet Gynecol Scand Suppl, V. 123, pp. 117-123, 1984.

Korenman et al., J of Clinical Endocrin and Metabolism, V. 71, p. 963, 1990.

Macphee et al., Epilepsia, V. 29, pp. 468-475, 1988.

Potts et al., Drugs, V. 19, pp. 321-330, 1980.

Pugeat et al., J of Clinical Endocrin and Metabolism, V. 53, p. 69, 1981.

Rosenfeld et al., J of Clinical Endocrinology, V. 32, p. 717, 1971

Zurlo et al., Fertility & Sterility, V. 54, p. 64, 1990.

1. The method of treating hypogonadism in the adult male which comprises administering to the person an amount of 17.alpha.-pregn-4en-20-yno[2,3-d]isoxazol-17-ol or pharmaceutical composition thereof effective in reducing or eliminating hypogonadism in the adult male.

2. The method according to claim 1 wherein the 17.alpha.-pregn-4-en-20-yno[2,3-d]isoxazol-17-ol is administered in a pharmaceutical composition.

3. The method according to claim 2 wherein the pharmaceutical composition is in the form of an oral dosage.

4. The method according to claim 3 wherein the oral unit dosage is a capsule containing as active ingredient from about 10 milligrams to about 50 milligrams of 17.alpha.-pregn-4-en-20-yno[2,3-d]isoxazol-17-ol.

5. The method according to claim 4 wherein the capsule contains as active ingredient 10 milligrams, 25 milligrams or 50 milligrams of 17.alpha.-pregn-4-en-20-yno[2,3-d]isoxazol-17-ol.

6. The method according to claim 5 wherein the capsule contains as active ingredient 25 milligrams of 17.alpha.-pregn-4-en-20-yno[2,3-d]isoxazol-17-ol.

7. The method according to claim 6 wherein the capsule is administered from one to four times daily.

8. The method according to claim 7 wherein the capsule is administered three times daily.

9. The method according to claim 2 wherein the pharmaceutical composition comprises a topical drug delivery system and a therapeutically effective amount of danazol retained therein for treating hypogonadism.

10. The method according to claim 9 wherein the topical pharmaceutical composition of danazol comprises a matrix base topical delivery system and an effective amount of danazol retained therein, and testosterone or other androgenic supplements.

11. The method of treating hypogonadism in the adult male which comprises administering to the person an amount of norgestrol (a therapeutic similar to danazol in its efficient displacement of bound testosterone from SHBG in the hypogonadal male) or pharmaceutical composition thereof effective in reducing hypogonadism in the adult male.

12. The method of treating hypogonadism in the adult male which comprises administering to the person an amount of 2-methoxyestradiol (a therapeutic similar to danazol in its efficient displacement of bound testosterone from SHBG in the hypogonadal male) or pharmaceutical composition thereof effective in reducing hypogonadism in the adult male.
